(19) United States
(12) Patent Application Publication
(10) Pub. No.: US 2006/0029570 A1
(43) Pub. Date: Feb. 9, 2006

(54) Therapeutic PEG solution concentrate

(75) Inventors: Bruce Howard Aronson, Sharon, MA (US); Stanley Edward Gay, Bridgewater, MA (US)

Correspondence Address: WILMER CUTLER PICKERING HALE AND DORR LLP, 60 STATE STREET, BOSTON, MA, 02109, US

(73) Assignee: Braintree Laboratories, Inc., Braintree, MA (US)

(21) Appl. No.: 10/990,796

(22) Filed: Nov. 17, 2004

Related U.S. Application Data
(60) Provisional application No. 60/523,200, filed on Nov. 17, 2003.

(51) Int. Cl. A61K 31/765

(52) U.S. Cl. 424/78.38

(57) ABSTRACT

The present invention provides a polyethylene glycol liquid concentrate for treatment of constipation or for gastrointestinal lavage that is chemically stable and resistant to microbial contamination.

### CROSS REFERENCE TO RELATED APPLICATIONS

This application claims priority under 35 U.S.C. § 119(e) to co-pending U.S. application Ser. No. 60/523,200, filed Nov. 17, 2003, and entitled “Therapeutic PEG Solution Concentrate,” which is incorporated in its entirety by reference.

### BACKGROUND

1. Field of the Invention

The present invention relates to the field of gastroenterology. More specifically, this invention relates to laxatives and laxative-based treatments and gastrointestinal (GI) lavages containing a concentrated liquid polyethylene glycol solution.

2. Description of the Related Art

Constipation is a gastrointestinal disorder characterized by a collection of symptoms defined by the international Rome II criteria (e.g., straining at defecation; lumpy or hard stools with defecation; and less than three defecations per week). Constipation is the most common gastrointestinal complaint in the United States; over 40,000,000 people (approximately 15-20% of the population) have frequent constipation as determined by self-assessment surveys.

Current treatments of constipation fall into two main categories, each with distinct disadvantages. One category, which includes the cathartics or purgatives and osmotic agents, causes an obligatory bowel movement to occur generally within minutes to a few hours, in an uncontrollable fashion. The bowel movement due to a cathartic or purgative laxative is characterized by unpredictability and urgency so that the patient's control of when or where the bowel movement occurs is virtually nonexistent. Examples of such cathartic laxatives include bisacodyl, senna, lactulose, saline laxatives, and gastrointestinal (GI) lavages.

A second category of laxatives, made up of so-called “bulk formers,” is composed of digestible or indigestible polymers of carbohydrates and/or other materials chemically synthesized or appearing in nature, such as psyllium and methyl cellulose. While the bulk formers do not produce a sense of uncontrollable urgency, the time course of their efficacy is longer in duration than the cathartics or purgatives, sometimes being as long as two to three days. Thus, while the sense of urgency is therefore diminished, the relief is delayed.

A better means of treating constipation combines the short time course of efficacy of a purgative with the lack of uncontrollable urgency that accompanies the bulk formers. Such a product produces overnight relief without urgency, and allows the patient to more readily control the time and place of their bowel movement, providing unique relief to their constipation syndrome.

Polyethylene glycol (“PEG”) is a currently marketed drug product prescribed by physicians for occasional constipation. The drug product includes PEG 3350 in the form of a white powder, as the active ingredient. As currently used, the patient dissolves a heaping tablespoon of the PEG powder (about 17 g) in an 8 oz. glass of water, juice, coffee, tea, soda, or other beverage choice to make one dose. Typically, the patient repeats the dosing once per day. Dilute solutions of PEG in water and other liquids (such as 17 g per 250 ml). Such dilute solutions may support microbial and bacterial growth, and thus, would not be chemically stable over the long periods of time required for a marketed product which must have a shelf life greater than at least six months. In addition, the weight of the large volume of solution would cause product shipping costs to be unacceptably expensive. Also, PEG in powder form requires about three minutes to dissolve in solution. Patients often complain about the time (about three minutes) required for the powder to dissolve in a solution.

### SUMMARY OF THE INVENTION

It has been discovered that concentrated solutions of polyethylene glycol are chemically stable and do not support microbial growth. These solutions can conveniently be used for consumption or for the preparation of a therapeutic solution for the treatment of constipation or for lavage. One advantage of the concentrated polyethylene glycol solution is that the powdered form of the polymer is already dissolved in an aqueous medium for the patient and further dilution is instantaneous. Patients can either administer the solution in its concentrated form or can dilute the polyethylene glycol concentrate in the liquid of their choice and then administer it to themselves.

This discovery has been exploited to develop the present invention, which in one aspect includes a composition comprising a shelf-stable and microbially-resistant therapeutic solution comprising an aqueous polyethylene glycol concentrate.

As used herein the term “concentrate” when used in connection with polyethylene glycol means a solution that is hyper-osmotic as compared to normal human plasma. Hyper-osmotic refers to a solution with a measured osmolarity of greater than 280 mOsm. The term “polyethylene glycol” encompasses polymers of polyethylene oxide (PEO) and polymers of polyoxyethylene (POE). The term “polymer” as used herein refers to a long, repeating chain of monomeric structural units. The monomeric structural units can be identical, or they can be different. The terms “PEO” and “POE” are understood to include branched and straight chain polymers. The term “shelf-stable” refers to the physical property of maintaining at least about 80% of its therapeutic effectiveness for at least two years at room temperature. The term “room temperature” refers to 25° C. at 60% relative humidity. The term “microbial-resistant” refers to the characteristic of not being susceptible to contamination by, or able to support the growth of, microorganisms such as, but not limited to, bacteria and yeast.

In some embodiments, the polyethylene glycol has an average molecular weight greater than about 1,000 Daltons to about 20,000 Daltons. In another embodiment, the polyethylene glycol has an average molecular weight ranging from about 1,500 Daltons to about 20,000 Daltons. In a further embodiment, the polyethylene glycol has an average molecular weight of about 3,000 Daltons to about 8,000 Daltons. In a particular embodiment, the polyethylene glycol has an average molecular weight of 3350 Daltons.

The composition can comprise from about 0.1 g to about 0.8 g polyethylene glycol per ml of solution. Alternatively, the composition comprises about 0.6 g polyethylene glycol per ml per dose. In some embodiments, the composition comprises from about 5 g to about 500 g polyethylene glycol per dose. In certain embodiments, the composition is provided in a form that is liquid, frozen, and/or incorporated into foodstuffs.

In some embodiments, the composition further comprises additional additives such as electrolytes and/or a stimulant laxative and/or a sweetener, a flavoring, a stabilizer, and/or a preservative.

### DESCRIPTION OF THE INVENTION

A chemically stable, microbial-resistant, aqueous polyethylene glycol concentrate has been devised for the treatment of constipation or for cathartic lavage. Because polyethylene glycol powder is granular and requires three or more minutes to dissolve in a liquid, patients benefit from having a pre-mixed solution of polymer. Such a solution can conveniently and easily be mixed into another vehicle or solution, or may be consumed as is. It may also be consumed in concentrated form in smaller volumes than it is typically consumed when in diluted forms.

Any food- or pharmaceutical-grade polyethylene glycol may be employed in the compositions contemplated herein. For example, polyethylene glycol polymers are commercially available (e.g., from The DOW Chemical Company, Midland, M.I., BASF Corporation, Mount Olive, N.J., or Clariant, Bergsen, Germany, or other vendor of food/pharmaceutical grade chemicals). PEG polymers of relatively high molecular weight (e.g., above about 1,500 Daltons) that are solid at room temperature (i.e., about 25° C.) and soluble in or miscible with water at room temperature are useful. PEG polymers having an average molecular weight of about 1,500 Daltons to about 20,000 Daltons, or between about 3,000 Daltons and about 8,000 Daltons are useful, such as, for example, PEG 3350, which has an average molecular weight of 3,350 Daltons.

Aqueous polyethylene glycol concentrates according to the present invention are prepared by dispersing and/or dissolving the polymer in water or other aqueous medium. Other aqueous media include, but are not limited to, juices, carbonated and other soft drinks, saline solutions, coffee, tea, milk and dairy products. The resulting polyethylene glycol concentrate can be a clear, colorless, generally tasteless and odorless liquid, formulated to a polymer concentration of about 0.1 g/ml to about 0.8 g/ml. The concentrate may be characterized as a syrup because it can be more viscous than water. The concentration of polyethylene glycol can be decreased or increased, the solubility of the polymer in water or the aqueous solution at room temperature being a limiting factor. Although higher concentrations of polyethylene glycol can (e.g., from Warner-Jenkinson, St. Louis, Mo., or other vendor of food/pharmaceutical grade colors). Preservatives can be added to keep freshness. Some useful preservatives include, but are not limited to, parabens, benzoates, sorbates, and alcohols, commercially obtainable (e.g., from Spectrum Quality Products, New Brunswick, N.J., or other vendor of food/pharmaceutical grade chemicals). The solution may be clear or unclear (cloudy, a suspension, etc.) with additives for product effect to look like orange juice, iced tea, and other drinks. Other additives can be used and the formula modified to optimize taste, odor, stability, solubility, acidity, color, etc. (see, e.g., U.S. Pat. Nos. 6,610,336 and 6,444,198).

The solution may also be prepared with other laxative products such as fiber bulking agents or stimulant laxatives. Useful fiber bulking agents include psyllium seed husk (available from e.g., Sarcom Distribution Center, Saratoga Springs, N.Y.), methyl cellulose (available from e.g., Aqualon Co., Hopewell, Va.) and polycarbophil (available from e.g., Boehringer Ingelheim Chemicals Inc, Petersburg, Va.). Useful stimulant laxatives include bisacodyl(available from e.g., Ohm Labs, North Brunswick, N.J., or other vendor of food/pharmaceutical grade stimulant laxatives). Polyethylene glycol, alone or in combination with one or more of sodium chloride, potassium chloride, potassium sulfate, sodium phosphate, phosphoric acid, and magnesium citrate may be used in the invention. The formulation may be a semi-solid, frozen, prepared as a chilled slurry or desert drink, or may be added to foods and other confections such as candies, as a topping, or as an ingredient in some other edible mixture.

Useful nonlimiting formulations of the polyethylene glycol concentrate of the invention are described below.

Formulation 1: 75 g PEG 3350, 490 ml purified water, 2 g sodium benzoate, 10.2 g citric acid, 1.9 ml Splenda, 8.3 ml Cherry flavor.

Formulation 2: 555 g PEG 3350, 500 ml purified water.

Formulation 3: 555 g PEG 3350, 500 ml purified water, 2 g sodium benzoate.

Formulation 4: 555 g PEG 3350, 490 ml purified water, 2 g sodium benzoate, 10.2 g citric acid, 1.9 ml Splenda, 8.3 ml Cherry flavor.

Formulation 5: 555 g PEG 3350, 490 ml purified water, 2 g sodium benzoate, 10.2 g citric acid, 1.9 ml Splenda, 8.3 ml Cherry flavor, 1.5 g Red #40.

Formulation 6: 555 g PEG 3350, 490 ml purified water, 2 g sodium benzoate, 10.2 g citric acid, 1.9 ml Splenda, 8.3 ml Tea flavor.

Formulation 7: 555 g PEG 3350, 490 ml purified water, 2 g sodium benzoate, 10.2 g citric acid, 1.9 ml Splenda, 8.3 ml Tea flavor, 1.7 g Caramel color.

Formulation 8: 555 g PEG 3350, 490 ml purified water, 2 g sodium benzoate, 10.2 g citric acid, 1.9 ml Splenda, 8.3 ml Grape flavor.

Formulation 9: 555 g PEG 3350, 490 ml purified water, 2 g sodium benzoate, 10.2 g citric acid, 1.9 ml Splenda, 8.3 ml Grape flavor, 0.13 g Purple color.

Formulation 10: 555 g PEG 3350, 490 ml purified water, 2 g sodium benzoate, 10.2 g citric acid, 1.9 ml Splenda, 8.3 ml Lemonade flavor.

Formulation 11: 555 g PEG 3350, 490 ml purified water, 2 g sodium benzoate, 10.2 g citric acid, 1.9 ml Splenda, 8.3 ml Lemonade flavor, 0.08 g Yellow Allum #5 and trace of blue color.

Formulation 12: 555 g PEG 3350, 500 ml purified water, and 20 grams of psyllium husk.

Formulation 13: 555 g PEG 3350, 500 ml purified water, and 96 g of magnesium citrate.

Formulation 14: 600 g PEG 3350, 490 ml purified water, 2 g sodium benzoate, 10.2 g citric acid, 1.9 ml Splenda, 8.3 ml Lemonade flavor.

The polyethylene glycol solution may be used for the treatment of children, adults, and geriatric patients as per their physician. With appropriate dose adjustments by veterinarians, it may be used for the treatment of animals.

Patients may ingest from about 0.1 tablespoon to about 50 tablespoons either in the concentrated form or conveniently diluted in from about 6 fluid oz. to about 10 fluid oz. (i.e., about 10-12 times the weight of the solid polyethylene glycol) of water, up to about four times per day as necessary for relief of symptoms. In other embodiments the patients may ingest from about 1 tablespoon to about 5 tablespoons of the concentrate either in concentrated form or diluted as described above. When used herein the term “dilute” means to make less concentrated by mixture of the therapeutic polyethylene glycol concentrate with a liquid.

To prepare a typical diluted dose of the polyethylene glycol, the patient mixes about 1.0 oz. (about 2 tablespoons) of solution with water to make about 8 oz. total in a glass. Alternatively, the syrup may be consumed without dilution, thereby reducing the volume needed as a laxative from about 8 oz. to about 1.0 oz. A glass of water or other drink following direct consumption of the syrup would then be recommended as a chaser. As a GI lavage, consumption of the syrup directly without dilution would reduce the volume required from about 128 oz. to less than about 16 oz., excluding the water chaser. Use of the solution improves patient compliance. Similar improvement is found if the solution is used as a GI lavage. Because the polyethylene glycol is an osmotically active agent that is not significantly absorbed from the gut, and may therefore be taken in dosages ranging from about 5 g to about 200 g up to four times per day, anywhere from about 10 g to about 30 g (depending on symptom severity) of polyethylene glycol in solid form are used to treat constipation.

Preparation of the liquid concentrate eliminates many of the packaging problems associated with a powder filling operation, which consists of a manual or automated procedure in which weighed amounts of a powder are added to a container. Such procedures are typically expensive, time-consuming, inaccurate and prone to error and waste. In the present invention the solution requires a liquid filling operation, which is convenient and rapid by comparison. Additionally, preparation of the concentrate takes up less space than a polyethylene glycol powder diluted to laxative concentration. The formulation can be considered to conserve energy and resources as the concentrated syrup saves on transportation costs. Also, the syrup can withstand a short period of high temperature exposure such as those which are known to melt powdered polyethylene glycols and form an unusable solid mass upon cooling.

The polyethylene glycol solution of the present invention may be used in much larger doses as a preparation for cleansing the bowel for diagnostic or operative purposes (e.g., as a gastrointestinal lavage preparation with or without supplemental electrolytes). For example, about 16 ounces (or an amount as prescribed by the patient's physician) may be used for cathartic purposes. About half the dose may be used when combined other laxatives such as Bisacodyl tablets in a gastrointestinal preparation. Electrolytes can be added if, for example, the formulation is used as a lavage or in other cases where electrolytes are needed by the patient. Useful electrolytes include sodium and potassium salts of chlorides, bicarbonates, sulfates, carbonates, and citrates. The concentrations of these electrolytes are dependent on the dose of laxative, and the need for obtaining electrolyte balancing of the patient's physiology. Nonlimiting examples of electrolyte concentrations that can achieve electrolyte balance are: sodium, 65-125 mmol/l, sulfate, 2040 mmol/l, chloride, 35-50 mmol/l, bicarbonate, 10-30 mmol/l and potassium, 5-10 mmol/l. Exemplary electrolytes can be commercially obtained (e.g., from Morton Salt, Mallinckrodt, St. Louis, Mo.; Spectrum Quality Products of New Brunswick N.J., or other vendors of food/pharmaceutical grade chemicals).

The foregoing description of the illustrative embodiments reveals the general nature of the method. Others of skill in the art will appreciate that applying ordinary skill may readily modify, or adapt, the method disclosed without undue experimentation. The descriptions of the illustrative embodiments are illustrative, not limiting. The method has been described in detail for illustration. Variations to the specific details can be made by those skilled in the art. For example, descriptions of a class or range useful include a description of any sub range or subclass contained therein, as well as a separate description of each member, or value in said class:

What is claimed is:

1. A composition comprising a shelf stable and microbially-resistant therapeutic solution comprising an aqueous polyethylene glycol concentrate.

2. The composition of claim 1, wherein the polyethylene glycol has an average molecular weight greater than about 1,000 Daltons to about 20,000 Daltons.

3. The composition of claim 2, wherein the polyethylene glycol has an average molecular weight ranging from about 1,500 Daltons to about 20,000 Daltons.

4. The composition of claim 3, wherein the polyethylene glycol has an average molecular weight ranging from about 3,000 Daltons to about 8,000 Daltons.

5. The composition of claim 4, wherein the polyethylene glycol is PEG 3350.

6. The composition of claim 1, wherein the solution comprises from about 0.1 g to about 0.8 g polyethylene glycol per ml of solution.

7. The composition of claim 1, wherein the solution comprises about 0.6 g/ml polyethylene glycol per dose.

8. The composition of claim 1, wherein the solution comprises from about 5 g to about 500 g polyethylene glycol per dose.

9. The composition of claim 1, further comprising electrolytes.

10. The composition of claim 1, which is provided in a form that is liquid, frozen, and/or incorporated into foodstuffs.

11. The composition of claim 1, further comprising a stimulant laxative.

12. The composition of claim 1, further comprising a sweetener.

13. The composition of claim 1, further comprising flavorings, stabilizers, and/or preservatives.

14. The composition of claim 1, further comprising fiber.

15. A method of treating constipation in a patient in need thereof, the method comprising administering to the patient the composition of claim 1.

16. A method of treating constipation in a patient in need thereof, the method comprising administering to the patient the composition of claim 5.

17. A method of treating constipation in a patient in need thereof, the method comprising:
a) diluting the composition of claim 1; and
b) administering the diluted composition to the patient.

18. A method for effecting gastrointestinal lavage in a patient in need thereof, the method comprising administering to the patient the composition of claim 1.

19. A method for effecting gastrointestinal lavage in a patient in need thereof, the method comprising administering to the patient the composition of claim 5.

20. A method for effecting gastrointestinal lavage in a patient in need thereof, the method comprising:
a) diluting the composition of claim 1; and
b) administering the diluted composition to the patient.
